(19) United States
(12) Patent Application Publication
(10) Pub. No.: US 2004/0023209 A1
(43) Pub. Date: Feb. 5, 2004

(54) Method for identifying microorganisms based on sequencing gene fragments

(76) Inventor: Jon Jonasson, Linkoping (SE)

Correspondence Address: DORSEY & WHITNEY LLP, INTELLECTUAL PROPERTY DEPARTMENT, 250 PARK AVENUE, NEW YORK, NY, 10177, US

(21) Appl. No.: 10/303,199

(22) Filed: Nov. 25, 2002

Related U.S. Application Data
(60) Provisional application No. 60/333,864, filed on Nov. 29, 2001.

(30) Foreign Application Priority Data
Nov. 28, 2001 (CA) 2,363,938

(51) Int. Cl. C12Q 1/70; C12Q 1/68

(52) U.S. Cl. 435/5; 435/6

(57) ABSTRACT

The present invention relates to a method of identifying a microorganism in a sample, based upon sequencing, and analysing, using a sequencing-by-synthesis procedure, short stretches, or fragments of a gene.

Accordingly, the present invention provides a method of identifying a microorganism in a sample, said method comprising:

determining the sequence of a region of up to 50 nucleotides in a predetermined site in a gene of said microorganism, thereby to obtain a signature sequence; and

analysing sequencing information in said signature sequence to identify said microorganism,

wherein said sequence is determined by detecting the nucleotides incorporated in a primer extension reaction performed using a primer binding at a pre-determined site in said gene.

### CROSS-REFERENCE TO RELATED APPLICATION

This application claims priority of U.S. application Ser. No. 60/333,864, the disclosure of which is incorporated herein by reference.

### BACKGROUND OF THE INVENTION

Microbial infections, namely the infection of a host organism by a microorganism, are one of the major causes of morbidity in general populations. In order to make an effective diagnosis of the disease or infection and to determine an appropriate treatment, it is important to identify rapidly and accurately the etiologic (i.e. causative) agent of the infection, namely to identify (or “type”) the microorganism involved in the infection.

In epidemiology, species information is also extremely important to determine the source and mode of transmission.

Conventional methods of diagnosing or typing microbial infections involve culturing a sample taken from the patient (e.g. blood sample), and re-culturing on selective growth medium. Biochemical characterization of the microorganism involved may then take place. Suitable methods of biochemical characterization include gram staining, colonial morphology, indole production testing and O-F reaction (testing whether an organism utilises glucose fermentively, oxidatively, or not at all) and other tests. These assays result in the identification of the species of microorganism involved in the infection, and provide no further information regarding the infection. The problems with conventional methods of typing microorganisms are multiple and can severely hinder prompt diagnosis of infection. Culturing microorganisms can be time-consuming, especially when the organism is slow growing or even non-cultivatable. For newer species there is a lack of accurate methods for typing.

Classical identification methods based on biochemical, serological, morphological and phenotypic characteristics are traditionally used to identify microorganism infections. However, as more information becomes available regarding microorganisms at the genetic level, the emphasis of diagnostic studies is shifting towards molecular methods, particularly those based on detection and analysis of nucleic acids, or genes, such as sequencing of the 16S rRNA (ribosomal RNA) genes of bacteria or the RNase P RNA gene. One advantage of molecular biology based identification or typing of microorganisms is that there is no need to culture samples. However, conventional sequencing methods used for typing (such as pulse field electrophoresis, hybridization or gel-based sequencing) can be time consuming, days or weeks may be required, and some methods are difficult to perform. Thus, even though nucleic acid sequence analysis is increasingly used for research purposes it is still considered too costly and time-consuming for use in large-scale molecular identification of microorganisms in a routine clinical diagnostic laboratory setting.

Identification of the species of microorganism involved in the infection does not always provide all the information required for the diagnosis, treatment and/or prognosis of the infection in the patient.

For accurate diagnosis, it would be advantageous not only to determine the general “class” (or genus or species) of infecting microorganism present, but also to determine which of the sub-types (e.g. strains) is present. For many infections, the infecting microorganism may occur in a number of different sub-types (strains or genotypes). The advantage of using molecular biology based techniques is that the sub-type (strain or genotype) of the infection microorganism can be identified. Molecular biology based analysis of the microorganism involved in the infection thus offers some advantages over standard techniques.

A need thus exists not only for a method which offers accurate and quick nucleic acid analysis and hence diagnosis of the infection, but which can be applied to a high number of samples in a high throughput setting in a cost-effective manner. Such information is vital, especially with life-threatening infections and epidemics of infection. Furthermore, such a method which may allow not only genus identification, but also species and strain typing information to be obtained would be highly advantageous. The present invention addresses this need.

The invention is thus based on deriving typing, or identification, information from relatively short nucleotide sequences contained in microorganism genes. This sequence information is derived using particular sequencing protocols which rely on specific priming and detection of the event of nucleotide incorporation (or non-incorporation) in such specific primer extension reactions. Such sequencing techniques enable valuable and discriminatory sequence information to be obtained from only short nucleotide sequences.

The potential of using genes, particularly the RNA genes and in particular the bacterial 16S rRNA gene or the RNase P RNA gene, as taxonomic tools have become increasingly evident in recent years. RNase P RNA is part of a ribonucleoprotein, Rnase P which is responsible for the maturation of the 5′-termini of tRNA molecules. The RNA subunit is approximately 400 nucleotides in length and is responsible for the catalytic activity of the RNase P. At the nucleotide level there are 4 regions of hypervariable nucleotide sequence known as the P3, P12, P17 and P19 loops. In bacteria, the major interspecies differences can be found in the P3 and P19 loops. The remaining ‘core structure’ which is thought to be essential for catalysis, is conserved across different species. The utility of the variable regions in detecting pathogenic organisms is discussed in WO01/51662.

The 16S rRNA is a structural part of the 30S ribosomal small subunit, whose functions are essential in the living cell. At the nucleotide level 16S rRNA consists of eight highly conserved regions, U1-U8, which are invariant across the bacterial domain. In between those conserved regions, nine variable regions can be distinguished, V1-V9, which are presumed to be segments of less importance for ribosomal function. These regions show a spectrum of different nucleotide substitution rates, which forms a favourable basis for phylogenetic analysis; the expression “rRNAs, the ultimate molecular chronometer” has been coined. Depending on species, bacterial chromosomes carry from 1 to 15 copies of rRNA genes. The individual rrn operons are monophyletic but heterogeneous 16S rRNA genes within a single microorganism are not rare. It is generally agreed that 16S rRNA and other ribosomal gene sequences are an unusually stable genotypic feature. Tens of thousands such molecules have been catalogued with sequences, structures and taxonomy in public molecular databases, e.g. GenBank at NCBI (http:///www.ncbi.nlm.nih.gov/). It has been proposed that these data can advantageously be used for identifying unknown bacteria by 16S rRNA gene sequencing (Relman, D. A., Schmidt, T. M., MacDermott, R. P. O and Falkow, S., 1992, New Engl. J. Med., 327, 293-301). However, as mentioned above such sequencing involved the use of conventional sequencing techniques, with all their attendant drawbacks, to sequence relatively long gene fragments making them unsuitable for use in a clinical diagnostic setting. However, we have now shown that highly accurate provisional classification or identification of commonly encountered clinically important bacteria and other micro-organisms can be obtained on a large scale using a sequencing-by-synthesis based technique for real-time DNA sequence analysis to obtain and analyse the sequence information content of that “signature” nucleotide sequences of selected gene sequences. This concept of “signature matching” is described further below.

Automated microbial identification in a clinical setting generally requires a fast and reliable, generally applicable identification system for approximately 1000 different, but sometimes closely related, pathogens. Most molecular diagnostic kits are narrow in scope and could not possibly fulfil this requirement. However, we have shown that a genotyping method of the present invention as described above, enables such analyses and is sufficiently discriminative to allow the rapid molecular identification, and even subtying, of a range of clinically important bacteria.

### BRIEF SUMMARY OF THE INVENTION

The present invention relates to a method of identifying a microorganism in a sample, advantageously a clinical sample, based upon sequencing, and analysing, using a sequencing-by-synthesis procedure, short stretches, or fragments of a gene.

Accordingly, the present invention provides a method of identifying a microorganism in a sample, said method comprising:

determining the sequence of a region of up to 50 nucleotides in a predetermined site in a gene of said microorganism, thereby to obtain a signature sequence; and

analysing sequencing information in said signature sequence to identify said microorganism,

wherein said sequence is determined by detecting the nucleotides incorporated in a primer extension reaction performed using a primer binding at a pre-determined site in said gene.

### BRIEF DESCRIPTION OF THE DRAWINGS

FIG. 1 shows two panels. The upper panel shows sequence alignment of 16S rDNA variable V1 region of H. pylori isolates HP-HJM 1-25 and reference strains H. pylori 26695 and J99. Gaps indicate deletions, and dashes indicate positions at which the sequences were homologous to that of reference strain H. pylori 26695. Lineages A to F indicate six individual 16S rDNA V1 alleles (signature sequences) at positions 75 to 99 (E. coli nomenclature). The 16S rDNA broad-range sequencing primer pBR-V1/as corresponds to a consensus sequence between positions 120 and 100 of many clinically important bacteria.

The lower panel shows sequence alignment of the variable V3 region of H. pylori isolates HP-HJM 1-25, reference strain H. pylori 26695 (AE000620/644), H. pylori J99 (AE001534/56), and the type strain H. pylori CCUG 17878^{T}(U01331). Gaps indicate deletions, and dashes indicate DNA sequence homologies compared to the type strain. The HP-V3T/as sequencing primer corresponds to the sequence of type strain H. pylori CCUG 17874^{T}.

For clarity, the corresponding sequences of H. pylori-related strains H. heilmanii (Y18028), H. bilis (AF047847), H. hepaticus (L39122) and H. cholecystus (U46129) are included; and

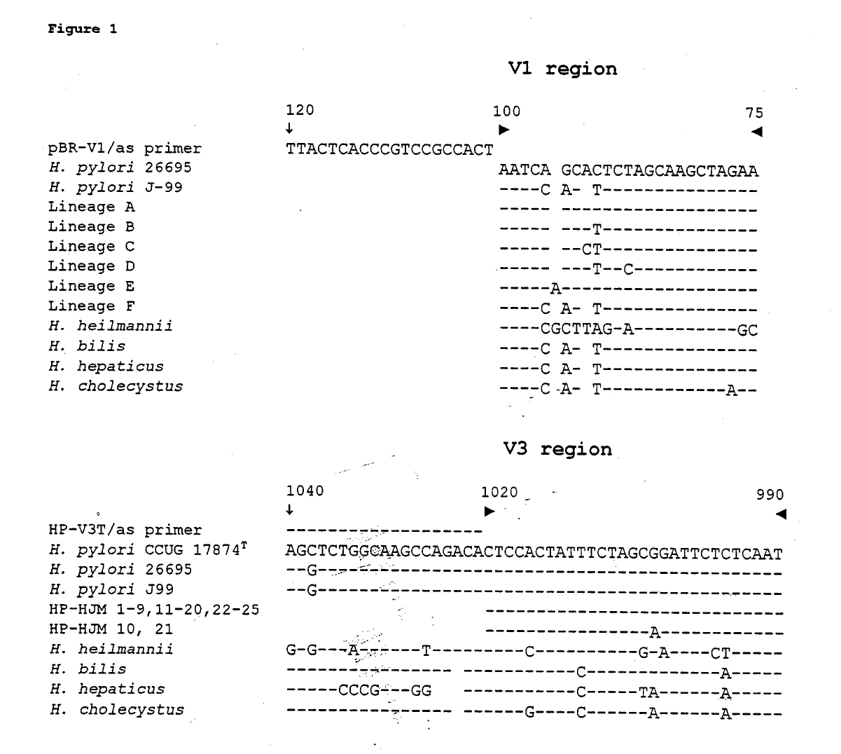

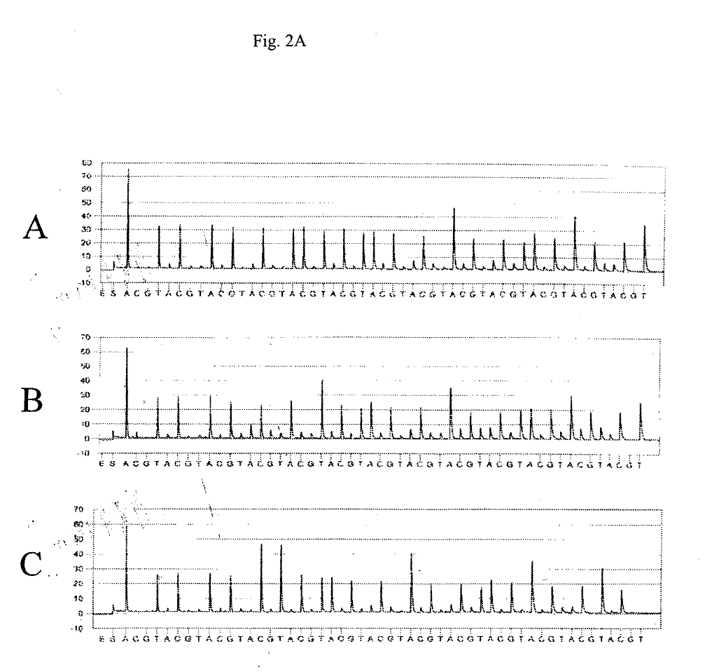

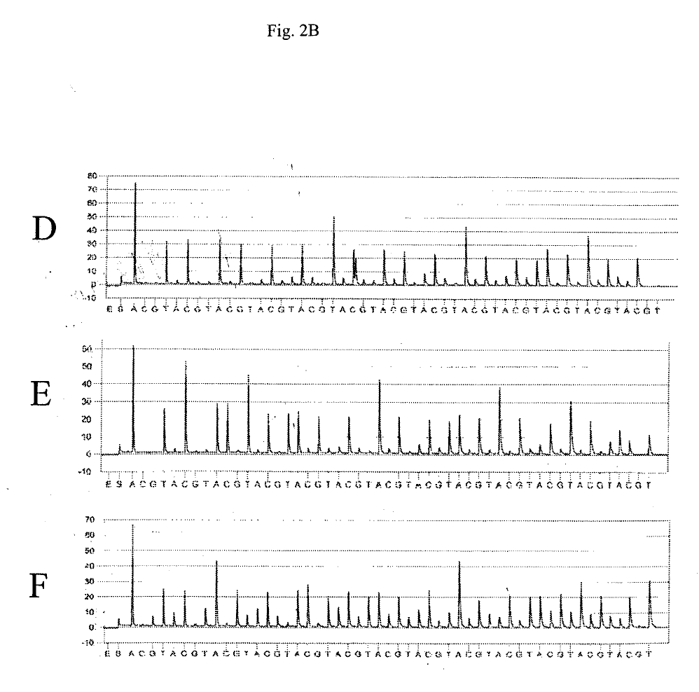

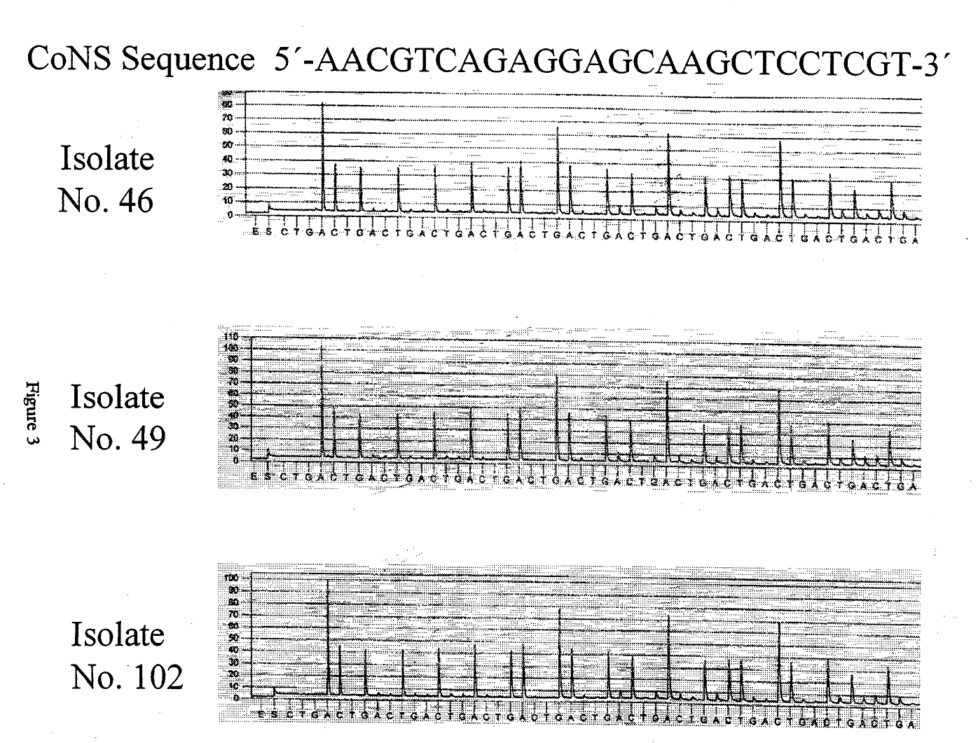

FIGS. 2A and 2B show Pyrosequencing™ of 16S rDNA variable V1 region of H. pylori isolates performed as described in Example 1 with cyclic dispensation of the nucleotides (Dispensation order: ACGT). Each pyrogram represents an individual H. pylori lineage (A-F). The corresponding nucleotide signature sequences as interpreted by a custom-made application program are shown in FIG. 1 (upper panel). The plots show nucleotide addition versus light emitted.

FIG. 3 shows Pyrosequencing™ obtained for 3 isolates obtained with the CoNS sequence 5-AACGTCAGAGGAGCAAGCTCCTCGT-3 using the pBR-V1 primer as the sequencing primer. The three isolates are coagulase negative staphylococci and appear to be three different isolates of staphylococcus epidemidis. The experimental method performed is as set out in Example 2. The Pyrograms™ shown are plotted as nucleotide added versus light emitted.

### DETAILED DESCRIPTION OF THE INVENTION

The term “identifying” as used herein includes all forms of detecting, determining and/or characterising the identity of the target microorganism. Thus, identify may be detected, determined or characterised at the genus, species, strain or particular genotype level, and different levels or degrees of information pertaining to the identity of the microorganism in question are encompassed by the present invention. The invention thus includes all methods of detecting a microorganism, and discriminating or distinguishing a microorganism. Thus, for example, the present invention allows pathogenic microorganisms to be distinguished from commensals or saprophytes in the same sample (e.g. in the same environment or habitat). Since sequence information is derived, the method of the invention permits molecular identification of microorganisms (e.g. microbial isolates) and hence it can be seen that genotyping may be achieved. The method of the invention thus includes methods of typing, and sub-typing, and classifying microorganisms. The methods of the invention may be used for general microorganism classification, characterisation, genotyping, epidemiological typing and phylogenetic analysis. The methods of the invention may particularly be used to ascribe an identity to (i.e. to identify) an unknown microorganism in a sample, including methods of provisional identification and provisional classification.

The “microorganism” according to the present invention may be any microorganism, and can be eukaryotic or prokaryotic. Such microorganisms are generally uni-cellular but need not be so limited. Advantageously however, the invention is performed on bacteria, which represent a significant class of microbial pathogens, although other organisms such as fungi, algae and protozoa are not excluded. The invention finds particular utility in the identification of pathogenic microorganisms.

The “sample” may be any sample or specimen which contains microorganisms and includes not only biological samples which may contain microorganisms e.g. samples of cellular or tissue material or body fluids, and microbial isolates or cultures, but also any cell cultures, suspensions or preparations, lysates, etc. which may contain microbial material, environmental samples (e.g. soil and water samples), food samples (e.g. from food manufacturers, caterers, restaurants, including testing utensils and cooking areas) etc. As mentioned above, the samples may contain microorganisms the identity of which is unknown. The samples may be freshly prepared or prior-treated in any convenient way e.g. for storage. Especially advantageously however, the sample will be a clinical sample, and this may thus include any tissue, cell or fluid sample which may be taken from a patient, to determine the presence or identity of a microbial infection. Representative samples include whole blood and blood-derived products such as plasma, serum and buffy coat, lymph, urine, cerebrospiral fluid, saliva, semen or any other body fluid, faeces tissues, biopsy samples or swabs. Microorganisms from such samples and specimen may be cultured, and such cultures may be used directly in the procedure e.g. a microbial cell suspension or other cell preparation or indeed a microbial colony (e.g. a bacterial colony). Alternatively, if desired nucleic acid may be extracted or isolated from the sample or microbial material in the sample.

The “patient” may be human, or a veterinary patient, such as farm animals including cattle, horses, sheep, pigs or chickens, companion animals such as dogs and cats, primates such as chimpanzees and gorillas, or any other animal. Herein, the term “animal” includes fish and birds.

It will be seen, therefore, that the method of the invention may be applied to any situation requiring the identification of a microorganism. Particularly advantageously, the method finds utility in identifying microorganisms in clinical samples, and hence in one aspect the invention can be seen as providing a method of diagnosis, for example, wherein the identity of a microbial pathogen causing an infection in a patient or subject is determined. However, the methods of the invention may equally be applied to any microbial classification study, e.g. phylogenetic or taxonomic studies, environmental monitoring, contamination testing, forensic analysis etc.

As explained above, the method involves sequencing a short stretch of nucleotides in a gene to obtain a “signature” sequence, the sequence information content of which may be used to identify the microorganism.

The gene may be any gene i.e. any gene encoding a product which may be an RNA molecule or a protein molecule. If the gene encodes a protein molecule, it will be understood that messenger mRNA is produced as an intermediate product.

Preferably the gene is an RNA gene. The RNA gene may be any RNA gene i.e. any gene encoding RNA as its final product. Such RNA genes include ribosomal RNA (rRNA) genes (e.g. 5S rRNA, 16S rRNa, 18S rRNA, 23S rRNA and 26S rRNA), transfer RNA (tRNA) genes, ribozymal RNA genes (e.g. the RNA component of RNase P) and the genes encoding the RNA components of telomerases, splicesomes and other RNA-protein complexes.

Preferably however, the gene will be a ribosomal RNA (rRNA) gene, namely a gene encoding a ribosomal RNA molecule. The rRNA may be of any ribosomal subunit, i.e. including both the large (50s) and small (30s) subunits. The rRNA may thus be the 16S molecule deriving from the 30s subunit or the 23S and 5S rRNAs deriving from the 50s subunit. Preferably, the rRNA gene is the 16S rRNA gene.

Alternatively however, the gene will encode a ribozyme RNA product, which may or may not associate with protein subunits to form a ribozyme. Preferably, the ribozyme RNA gene is the gene for RNase P RNA.

A surprising feature of the present invention is that sufficient sequence information to identify a microorganism may be derived from a relatively short nucleotide sequence, namely a sequence of not more than 50 nucleotides. Indeed it has been found that discriminatory information sufficient to identify a microorganism (e.g. at a provisional level or at genus level) may be obtained from a nucleotide sequence as short as 6 nucleotides, e.g., 10 nucleotides. Thus, the region sequenced may be from 6, 10, 12, 15, 20 or 25, nucleotides long, and up to e.g., 30, 35, 40, 45 to 50 nucleotides long and any combination derived therefrom, e.g. 6 to 50, 6 to 40, 10 to 40, 12 to 40, 15 to 40, 10 to 30, 10 to 25, 10 to 20 or 10 to 15, nucleotides long. In some cases a longer stretch may be sequenced e.g. 15 to 50, 20 to 50, 25 to 40 nucleotides etc. It is possible to combine different sequences from different regions to yield further discriminatory or identificatory information, and this may in certain cases enable shorter sequences to be used. Thus, the method of the invention may be performed by sequencing one or more (i.e. multiple) regions of up to 50 nucleotides of a gene e.g. 2, 3, 4, 5 or more e.g. 1 to 9 or 1 to 6 (e.g. 2 to 6) nucleotides. For example a region from each of the nine variable regions (V1 to V9) of the 16S rRNA gene may be sequenced, or a particular combination thereof, e.g. V1 and V3.

In order for the sequenced region to provide discriminatory information, it will be appreciated that it needs to be variable or distinguishable, as between different microorganisms.

It can thus be viewed as a “discriminatory” or “variable” region. As mentioned above, the sequenced region lies in a pre-determined site in the gene. Thus, the region may be selected to lie in or overlap with a region or site (or locus) of sequence variability (i.e. genetic variation), namely a site or region which is not conserved as between different microorganisms. As mentioned above, ribosomal genes contain regions of variability e.g. V1 to V9 for the 16S RNA gene, or P3, P12, P17 and P19 for the RNase P RNA gene, and such variable regions or sequences within them, may be used as the variable region according to the present invention.

On the other hand, in order to be able to obtain a primer extension product from a range of different microorganisms (i.e. from any microorganism which may be present in the sample) it will be understood that the primer needs to bind at site which is common (i.e. conserved or semi-conserved) as between different microorganisms. Thus, in order to perform the invention the primer binding site should be available in all individual microorganisms which may be present in the sample. Such primer binding sites will therefore advantageously lie in regions which are common to, or substantially conserved between different microorganisms. This may readily be achieved by selecting the primer binding site to lie in conserved/semi-conserved regions as discussed above. Thus, the extension primer (i.e. the sequencing primer) is designed or selected to bind at a pre-determined site which is common to (a conserved or semi-conserved) different microorganisms. Such a primer may be regarded as a universal primer i.e. a primer capable of binding to the selected gene of a range of different microorganisms i.e. of binding non-selectively insofar as the microorganism is concerned, although of course binding primer is specific as regards its binding site. Such conserved regions may e.g. be or lie within the regions U1 to U8 of the 16S rRNA gene or the conserved core structure of the RNase P RNA mentioned above. The primer is further designed or selected so that when the primer extension reaction is performed the primer is extended over the “variable” or “discriminatory” region to be sequenced. In other words, the extension primer is designed or selected so that its extension product overlaps (or comprises) a region of sequence variability. Thus, the primer binds to the target gene at, or near to, (e.g. within 1 to 40, 1 to 20, 1 to 10, or 1 to 6 bases of) a variable region or site. It will be seen therefore that primer binding sites may be selected which flank a variable region. Where more than one region is to be sequenced, two or more primers are provided, each binding at a different pre-determined site.

From the above it will be appreciated that to design or select the predetermined sites of the variable region and the primer binding site, knowledge of the sequence of the target gene is required.

Primers suitable for use as extension primers of the invention may be publically available, for example primers known for sequencing ribosomal genes e.g. pJBS-V3.SE, B-V3.A5 and pBR.-V1.A5 sequencing primers for V3 and V1 regions in the 16S rRNA gene (Monstein et al., 2001, FEMS Microbiology Letters, 199, 103-107 and Jonasson et al., APMIS 2002, March; 110(3): 263-72).

The sequencing, or primer extension, step results in the obtention of a “signature” sequence for the target microorganism. In other words, the sequence interpreted from detecting nucleotide incorporation in the primer extension step may be used as the signature of the gene of the target microorganism. This signature sequence may thus be viewed as an identificatory or characterising sequence or “tag” or “motif” for a microorganism. The signature sequence may contain a range of sequence information or data which may be used to identify the microorganism. This may include both full sequence information, identification of particular substitutions or base identity at defined positions (i.e. “landmark” sequence data), combinations or substitutions or of base identity at particular positions, uniqueness of the signature sequence or of base identity at particular positions within it, detection of matches and/or mismatches, insertions, deletions etc. Thus, a signature sequence may have multiple signature attributes.

The information content (i.e. sequence data) in the signature sequence is analysed to identify the microorganism. This analysis step may be accomplished in any known or desired manner for assessing or evaluating sequence information. Thus, the analysis may involve comparing the signature sequence obtained against one or more reference or standard sequences (e.g. a panel or catalogue or database of sequences or a consensus sequence or “template” sequence).

A reference or standard sequence may readily be obtained using publically available information, for example the rRNA sequences and sequence databases mentioned above, or by determining the sequence of one or more known genes using the same sequence procedure (e.g. the same extension primer) as the method of the invention.

The comparison may involve determining sequence identity or similarity using known procedures, comparing particular positions, substitutions, or other sequence features etc., determining the presence of matches, mismatches etc. Thus, a matching step may be performed, wherein it is determined whether or not the signature sequence, or any positions or combinations of positions within it, match a known sequence. Sequence alignments may be performed, again using known procedures. The pattern of nucleotide incorporation detected in the primer extension step may be analysed. Where multiple (i.e. 2 or more) signature sequences are obtained, the sequence information may be analysed combinatorially (e.g. aspects of particular sequence information, or particular attributes may be combined, or assessed together).

Alternatively, the “reference” sequence can be theoretically derived from knowledge of the selected variable region. It may then not be necessary actually to compare the signature sequence obtained with a reference sequence, and the desired typing/sequence information can be read from the sequence obtained. Once the extension primers for each variable region have been selected and the order of addition of nucleotides determined, it is possible to determine a theoretical output from a primer extension reaction. Thus, by identifying (or recognising) the sequence obtained for a target microorganism molecule may be identified (or recognised). Conveniently, test sequences or patterns and reference sequences or patterns may be compared using sequence recognition software. All such analysis procedures are regarded herein as a step of “matching” the signature sequence.

Such matching or analysis procedures may be performed in any convenient or desired manner, for example manually, or in an automated fashion using e.g. appropriate computer software (e.g. computer algorithms). Various software for sequence analysis is available publically, for example the BLAST advanced option tools available at NCBI (http://www.ncbi.nlm.nih.gov/).

As described further below, the present invention is based on a method of “sequencing-by-synthesis” (see e.g. U.S. Pat. No. 4,863,849 of Melamede). This is a term used in the art to define sequencing methods which rely on the detection of nucleotide incorporation (or non-incorporation) during a primer-directed polymerase extension reaction. The four different nucleotides (i.e. A, G, T or C nucleotides) are added cyclically or sequentially (conveniently in a known order), and the event of incorporation can be detected in various ways, directly or indirectly, This detection reveals which nucleotide has been incorporated, and hence sequencing information; when the nucleotide (base) which forms a pair (according to the normal rules of base pairing, A-T and C-G) with the next base in the template target sequence is added, it will be incorporated into the growing complementary strand (i.e. the extended primer) by the polymerase, and this incorporation will trigger a detectable signal, the nature of which depending upon the detection strategy selected.

The primer extension reaction in the sequencing step conveniently may be performed by sequentially adding nucleotides to the reaction mixture (i.e. a polymerase, and primer/template mixture). Advantageously the different nucleotides are added in known order, and preferably in a pre-determined order. In a convenient embodiment of the invention, the 4 different nucleotides (i.e. A, G, T and C nucleotides) are added sequentially in a predetermined order of addition. It thus forms a preferred aspect of the invention that the nucleotides are added sequentially in a predetermined order of addition. If desired, the order of addition can be tailored to the microorganism to be identified or to the ribosomal gene in question and the primers used. It will therefore be seen that the order of addition will not necessarily be cyclical e.g. A T G C A T G C but can be e.g. C G C T A G A. Indeed, it may not be necessary to add all our nucleotides, (i.e. all of A, T, C or G) but a desired selection thereof.

As each nucleotide is added, it may be determined whether or not nucleotide incorporation takes place.

Advantageously, as described in more detail below, it may further be determined the amount (i.e. how many) of each nucleotide incorporated. In this manner, the sequence or a pattern of nucleotide incorporation may be determined. In other words, the step of determining the sequence may comprise determining (or detecting) whether or not, and which, nucleotide is incorporated. If desired, this step also includes determining the amount of each nucleotide incorporated.

In this manner, a “signature” may be obtained for the target microorganism. This “signature” may comprise the base identity (i.e. sequence) of the particular variable sites identified in the variable region for that microorganism.

In order to perform the invention, it may be advantageous or convenient first to amplify the nucleic acid molecule by any suitable amplification method known in the art. The target region to be sequenced would then be an amplicon. Suitable in vitro amplification techniques include any process which amplifies the nucleic acid present in the reaction under the direction of appropriate primers. The amplification method may thus preferably be PCR, or any of the various modifications thereof e.g. the use of nested primers, although it is not limited to this method. Those skilled in the art will appreciate that other amplification procedures may also be used, such as Self-sustained Sequence Replication (3SR), NASBA, the Q-beta replicase amplification system and Ligase chain reaction (LCR) (see for example Abramson and Myers (1993) Current Opinion in Biotech., 4: 41-47).

If PCR is used to amplify the nucleic acid, suitable primers, as discussed previously, are designed or selected to ensure that the region of interest within the nucleic acid sequence (i.e. the variable region), is amplified. PCR can also be used for indiscriminate amplification of all DNA sequences, allowing amplification of essentially all sequences within the sample for study (i.e. total DNA). Linker-primer PCR is particularly suitable for indiscriminate amplification, and uses double stranded oligonucleotide linkers with a suitable overhanging end, which are ligated to the ends of target DNA fragments. Amplification is then conducted using oligonucleotide primers which are specific for the linker sequences. Alternatively, completely random oligonucleotide primers may be used in conjunction with DOP-PCR (degenerate oligonucleotide-primed) to amplify all the DNA within a sample. Preferably, however amplification is conducted using primers having binding sites which are common or conserved as between different organisms i.e. universal primers designed or selected along the principles set out above for the extension, or sequencing primer. Conveniently, broad-range amplification primers may be used.

In the method of the invention, several sequences may need to be amplified, to allow several regions to be analysed. Therefore, several appropriate amplification primers may need to be synthesized or selected.

In a preferred embodiment of the invention, one or more of the amplification primers used in the amplification reaction, may be subsequently used as an “extension primer” in the sequencing step. This has the advantage that an amplicon will always yield a primer extension product in the sequencing step. It will be appreciated that the sequence and length of the oligonucleotide amplification and extension primers to be used in the amplification and extension (sequencing) steps, respectively, will depend on the sequence of the target gene, the desired length of amplification or extension product, the further functions of the primer (i.e. for immobilization) and the method used for amplification and/or extension. Appropriate primers may readily be designed applying principles and techniques well known in the art.

Advantageously, as mentioned above, an extension primer will bind near (e.g. within 1-40, 1-20, 1-10 or 1-6, preferably within 1-3 bases), substantially adjacent or exactly adjacent to the variable region of the gene and will be complementary to a conserved or semi-conserved region of the gene. In order for the method of the invention to be performed, knowledge of the sequence of the conserved or semi-conserved region is required in order to design an appropriate complementary extension primer. An extension primer is provided for each of the variable regions, each being specific for a site at or near to the variable site. The specificity is achieved by virtue of complementary base pairing. For all embodiments of the invention, primer design may be based upon principles well known in the art. It is not necessary for the extension or amplification primer to have absolute complementarily to the binding site, but this is preferred to improve the specificity of binding.

The extension primer may be designed to bind to the sense or anti-sense strand of the target gene.

In a preferred embodiment of the invention, the extension primers are designed to bind to the target gene near to the variable region in such a way that upon the addition of nucleotides in a predetermined manner, the sequencing of particular positions or sites in the variable region or a particular variable region takes place discretely. The “primer extension” reaction according to the invention includes all forms of template-directed polymerase-catalysed nucleic acid synthesis reactions. Conditions and reagents for primer extension reactions are well known in the art, and any of the standard methods, reagents and enzymes etc. may be used in this step (see e.g. Sambrook et al., (eds), Molecular Cloning: a laboratory manual (1989), Cold Spring Harbor Laboratory Press). Thus, the primer extension reaction at its most basic, is carried out in the presence of primer, deoxynucleotides (dNTPs) and a suitable polymerase enzyme e.g. T7 polymerase, Klenow or Sequenase Ver 2.0 (USB USA), or indeed any suitable available polymerase enzyme. As mentioned above, for an RNA template, reverse transcriptase may be used. Conditions may be selected according to choice, having regard to procedures well known in the art.

The primer is thus subjected to a primer-extension reaction in the presence of a nucleotide, whereby the nucleotide is only incorporated if it is complementary to the base immediately adjacent (3′) to the primer position. The nucleotide may be any nucleotide capable of incorporation by a polymerase enzyme into a nucleic acid chain or molecule. Thus, for example, the nucleotide may be a deoxynucleotide (dNTP, deoxynucleoside triphosphate) or dideoxynucleotide (ddNTP, dideoxynucleoside triphosphate). Thus, the following nucleotides may be used in the primer-extension reaction: guanine (G), cytosine (C), thymine (T) or adenine (A) deoxy- or dideoxy-nucleotides. Therefore, the nucleotide may be dGTP (deoxyguanosine triphosphate), dCTP (deoxycytidine triphosphate), dTTP (deoxythymidine triphosphate) or dATP (deoxyadenosine triphosphate). As discussed further below, suitable analogues of dATP, and also for dCTP, dGTP and dTTP may also be used. Thus, modified nucleotides, or nucleotide derivatives may be used so long as they are capable of incorporating and including an activated or detectably-labelled nucleotides (e.g. radio or fluoroscently labelled nucleotide triphosphates for example, a suitable fluorescently labelled nucleotide triphosphate is cyanine 5 S-S-d NTP available from NEN Life Sciences, Boston, USA and as described in WO 00/53812). Dideoxynucleotides may also be used in the primer-extension reaction. The term “dideoxynucleotide” as used herein includes all 2′-deoxynucleotides in which the 3′ hydroxyl group is modified or absent. Dideoxynucleotides are capable of incorporation into the primer in the presence of the polymerase, but cannot enter into a subsequent polymerisation reaction, and thus function as a “chain terminator”. It will therefore be appreciated that in embodiments of the invention which rely on sequential nucleotide addition the use of chain terminating nucleotides is to be avoided (although so-called “false” or “labile” terminators might be used in which the 3′blocking group may be removed following incorporation. Such modified nucleotides are known and described in the art). However, in some embodiments of the invention it may be advantageous to use chain terminating nucleotides whereby it is desired to terminate sequencing of one variable region after incorporation of the chain terminating nucleotide, but more sequence information is required for another region.

If the nucleotide is complementary to the target base, the primer is extended by one nucleotide, and inorganic pyrophosphate is released. As discussed further below, in a preferred method, the inorganic pyrophosphate may be detected in order to detect the incorporation of the added nucleotide. The extended primer can serve in exactly the same way in a repeated procedure to determine the next base in the variable region, thus permitting the whole variable region to be sequenced. Different nucleotides may be added sequentially, advantageously in known order, as discussed above, to reveal the nucleotides which are incorporated for each extension primer. Furthermore, in the case where the variable region is homopolymeric or contains a homopolymer site (i.e. contains 2 or more identical bases), the number of nucleotides incorporated of the complementary base will reflect the number present in the homopolymeric region. Accordingly, determining the number of nucleotides incorporated for each nucleotide addition, will reveal this information.

Hence, a primer extension protocol may involve annealing a primer as described above, adding a nucleotide, performing a polymerase-catalysed primer extension reaction, detecting the presence or absence of incorporation of said nucleotide (and advantageously also determining the amount of each nucleotide incorporated) and repeating the nucleotide addition and primer extension steps etc. one or more times. As discussed above, single (i.e. individual) nucleotides may be added successively to the same primer-template mixture.

In order to permit the repeated or successive (iterative) addition of nucleotides in a primer-extension procedure, the previously-added nucleotide must be removed. This may be achieved by washing, or more conveniently, by using a nucleotide-degrading enzyme, for example as described in detail in WO98/28440.

Accordingly, in a principal embodiment of the present invention, a nucleotide degrading enzyme is used to degrade any unincorporated or excess nucleotide. Thus, if a nucleotide is added which is not incorporated (because it is not complementary to the target base), or any added nucleotide remains after an incorporation event (i.e. excess nucleotides) then such unincorporated nucleotides may readily be removed by using a nucleotide-degrading enzyme. This is described in detail in WO98/28440.

The term “nucleotide degrading enzyme” as used herein includes any enzyme capable of specifically or non-specifically degrading nucleotides, including at least nucleoside triphosphates (NTPs), but optionally also di- and mono-phosphates, and any mixture or combination of such enzymes, provided that a nucleoside triphosphatase or other NTP-degrading activity is present. Where a chain terminating nucleotide is used (e.g. a dideoxy nucleotide is used), the nucleotide degrading enzyme should also degrade such a nucleotide. Although nucleotide-degrading enzymes having a phosphatase activity may conveniently be used according to the invention, any enzyme having any nucleotide or nucleoside degrading activity may be used, e.g. enzymes which cleave nucleotides at positions other than at the phosphate group, for example at the base or sugar residues. Thus, a nucleoside triphosphate degrading enzyme is essential for the invention. Nucleoside di- and/or mono-phosphate degrading enzymes are optional and may be used in combination with a nucleoside triphosphate degrading enzyme.

The preferred nucleotide degrading enzyme is apyrase, which is both a nucleoside diphosphatase and triphosphatase, catalysing the reactions NTP NDP+Pi and NDP NMP+Pi (where NTP is a nucleoside triphosphate, NDP is a nucleoside diphosphate, NMP is a nucleotide monophosphate and Pi is inorganic phosphate). Apyrase may be obtained from the Sigma Chemical Company. Other possible nucleotide degrading enzymes include Pig Pancreas nucleoside triphosphate diphosphorydrolase (Le Bel et al., 1980, J. Biol. Chem., 255, 1227-1233). Further enzymes are described in the literature.

The nucleotide-degrading enzyme may conveniently be included during the polymerase (i.e. primer extension) reaction step. Thus, for example the polymerase reaction may conveniently be performed in the presence of a nucleotide-degrading enzyme. Although less preferred, such an enzyme may also be added after nucleotide incorporation (or non-incorporation) has taken place, i.e. after the polymerase reaction step.

Thus, the nucleotide-degrading enzyme (e.g. apyrase) may be added to the polymerase reaction mixture (i.e. target nucleic acid, primer and polymerase) in any convenient way, for example prior to or simultaneously with initiation of the reaction, or after the polymerase reaction has taken place, e.g. prior to adding nucleotides to the sample/primer/polymerase to initiate the reaction, or after the polymerase and nucleotide are added to the sample/primer mixture.

Conveniently, the nucleotide-degrading enzyme may simply be included in the reaction mixture for the polymerase reaction, which may be initiated by the addition of the nucleotide.

According to the present invention, detection of nucleotide incorporation can be performed in a number of ways, such as by incorporation of labelled nucleotides which may subsequently be detected.

As explained above, the invention uses a sequencing-by-synthesis method, and such methods are disclosed extensively in U.S. Pat. No. 4,863,849, which discloses a number of ways in which nucleotide incorporation may be determined or detected, e.g. spectrophotometrically or by fluorescent detection techniques, for example by determining the amount of nucleotide remaining in the added nucleotide feedstock, following the nucleotide incorporation step. In a sequencing-by-synthesis reaction, determination of the pattern of nucleotide incorporation may occur simultaneously with primer extension. One working definition of sequencing by synthesis is a method in which a single nucleotide is or is not incorporated into a primed template, incorporation being detected by any suitable means. This step is repeated by addition of a different nucleotide and incorporation is again detected. These steps are repeated and from the sum of incorporated nucleic acids the sequence can be deduced.

Thus, in the method of the invention it may be directly determined whether or not incorporation of a given nucleotide has taken place. Contrary to conventional sequencing methods (e.g. dideoxy sequencing), sequencing-by-synthesis allows the ordinal numbering of bases to be determined, and it is known exactly where the sequencing primer binds. Consequently, it is possible readily to derive position used sequence data or information (e.g. which bases are incorporated in which position). Conveniently, sequencing may start from either end of an amplicon.

One method of sequencing-by-synthesis is a method based on the detection of incorporation of fluorescently labelled nucleotides.

The preferred method of sequencing-by-synthesis is a pyrophosphate detection-based method.

Preferably, therefore, nucleotide incorporation is detected by detecting PPi release, preferably by luminometric detection, and especially by bioluminometric detection.

PPi can be determined by many different methods and a number of enzymatic methods have been described in the literature (Reeves et al., (1969), Anal. Biochem., 28, 282-287; Guillory et al., (1971), Anal. Biochem., 39, 170-180; Johnson et al., (1968), Anal. Biochem., 15, 273; Cook et al., (1978), Anal. Biochem. 91, 557-565; and Drake et al., (1979), Anal. Biochem. 94, 117-120).

It is preferred to use luciferase and luciferin in combination to identify the release of pyrophosphate since the amount of light generated is substantially proportional to the amount of pyrophosphate released which, in turn, is directly proportional to the amount of nucleotide incorporated. The amount of light can readily be estimated by a suitable light sensitive device such as a luminometer. Thus, luminometric methods offer the advantage of being able to be quantitative.

Luciferin-luciferase reactions to detect the release of PPi are well known in the art. In particular, a method for continuous monitoring of PPi release based on the enzymes ATP sulphurylase and luciferase has been developed (Nyrén and Lundin, Anal. Biochem., 151, 504-509, 1985; Nyrén P., Enzymatic method for continuous monitoring of DNA polymerase activity (1987) Anal. Biochem Vol 167 (235-238)) and termed ELIDA (Enzymatic Luminometric Inorganic Pyrophosphate Detection Assay). The use of the ELIDA method to detect PPi is preferred according to the present invention. The method may however be modified, for example by the use of a more thermostable luciferase (Kaliyama et al., 1994, Biosci. Biotech. Biochem., 58, 1170-1171) and/or ATP sulfurylase (Onda et al., 1996, Bioscience, Biotechnology and Biochemistry, 60:10, 1740-42). This method is based on the following reactions:

ATP sulphurylase

PPi+APS - - - >ATP+SO₄²⁻

luciferase

ATP+luciferin+O₂ - - - >AMP+PPi+oxyluciferin+CO₂+hv

(APS=adenosine 5′-phosphosulphate)

Reference may also be made to WO 98/13523 and WO 98/28448, which are directed to pyrophosphate detection-based sequencing procedures, and disclose PPi detection methods which may be of use in the present invention.

In a PPi detection reaction based on the enzymes ATP sulphurylase and luciferase, the signal (corresponding to PPi released) is seen as light. The generation of the light can be observed as a curve known as a Pyrogram™. Light is generated by luciferase action on the product, ATP (produced by a reaction between PPi and APS (see below) mediated by ATP sulphurylase) and, where a nucleotide-degrading enzyme such as apyrase is used, this light generation is then “turned off” by the action of the nucleotide-degrading enzyme, degrading the ATP which is the substrate for luciferase. The slope of the ascending curve may be seen as indicative of the activities of DNA polymerase (PPi release) and ATP sulphurylase (generating ATP from the PPi, thereby providing a substrate for luciferase). The height of the signal is dependent on the activity of luciferase, and the slope of the descending curve is, as explained above, indicative of the activity of the nucleotide-degrading enzyme. In a Pyrogram™ in the context of a homopolymeric region, peak height is also indicative of the number of nucleotides incorporated for a given nucleotide addition step. Thus, when a nucleotide is added, the amount of PPi released will depend upon how many nucleotides (i.e. the amount) are incorporated, and this will be reflected in the slope height.

Advantageously, by including the PPi detection enzyme(s) (i.e. the enzyme or enzymes necessary to achieve PPi detection according to the enzymatic detection system selected, which in the case of ELIDA, will be ATP sulphurylase and luciferase) in the polymerase reaction step, the method of the invention may readily be adapted to permit extension reactions to be continuously monitored in real-time, with a signal being generated and detected, as each nucleotide is incorporated.

Thus, the PPi detection enzymes (along with any enzyme substrates or other reagents necessary for the PPi detection reaction) may simply be included in the polymerase reaction mixture.

A potential problem which has previously been observed with PPi-based sequencing methods is that dATP, used in the chain extension reaction, interferes in the subsequent luciferase-based detection reaction by acting as a substrate for the luciferase enzyme. This may be reduced or avoided by using, in place of deoxyadenosine triphosphate (ATP), a dATP analogue which is capable of acting as a substrate for a polymerase but incapable of acting as a substrate for a PPi-detection enzyme. Such a modification is described in detail in WO98/13523.

The term “incapable of acting” includes also analogues which are poor substrates for the detection enzymes, or which are substantially incapable of acting as substrates, such that there is substantially no, negligible, or no significant interference in the PPi detection reaction.

Thus, a further preferred feature of the invention is the use of a dATP analogue which does not interfere in the enzymatic PPi detection reaction but which nonetheless may be normally incorporated into a growing DNA chain by a polymerase. By “normally incorporated” is meant that the nucleotide is incorporated with normal, proper base pairing. In the preferred embodiment of the invention where luciferase is a PPi detection enzyme, the preferred analogue for use according to the invention is the [1-thio]triphosphate (or -thiotriphosphate) analogue of deoxy ATP, preferably deoxyadenosine [1-thio]triphospate, or deoxyadenosine -thiotriphosphate (dATP S) as it is also known. dATP S, along with the -thio analogues of dCTP, dGTP and dTTP, may be purchased from Amersham Pharmacia. Experiments have shown that substituting dATP with dATP S allows efficient incorporation by the polymerase with a low background signal due to the absence of an interaction between dATP S and luciferase. False signals are decreased by using a nucleotide analogue in place of dATP, because the background caused by the ability of dATP to function as a substrate for luciferase is eliminated. In particular, an efficient incorporation with the polymerase may be achieved while the background signal due to the generation of light by the luciferin-luciferase system resulting from dATP interference is substantially decreased. The dNTP S analogues of the other nucleotides may also be used in place of the other dNTPs.

Another potential problem which has previously been observed with sequencing-by-synthesis methods is that false signals may be generated and homopolymeric stretches (i.e. CCC) may be difficult to sequence with accuracy. This may be overcome by the addition of a single-stranded nucleic acid binding protein (SSB) once the extension primers have been annealed to the template nucleic acid. The use of SSB in sequencing-by-synthesis is discussed in WO 00/43540 of Pyrosequencing AB.

In order for the primer-extension reaction to be performed, the nucleic acid molecule to the sequenced (i.e. the ribosomal gene), regardless of whether or not it has been amplified, is conveniently provided in a single-stranded format. The nucleic acid may be subjected to strand separation by any suitable technique known in the art (e.g. Sambrook et al., supra), for example by heating the nucleic acid, or by heating in the presence of a chemical denaturant such as formamide, urea or formaldehyde, or by use of alkali.

However, this is not absolutely necessary and a double-stranded nucleic acid molecule may be used as template, e.g. with a suitable polymerase having strand displacement activity.

Where a preliminary amplification step is used, regardless of how the nucleic acid has been amplified, all components of the amplification reaction need to be removed, to obtain pure nucleic acid, prior to carrying out the typing assay of the invention. For example, unincorporated nucleotides, PCR primers, and salt from a PCR reaction need to be removed. Methods for purifying nucleic aids are well known in the art (Sambrook et al., supra), however a preferred method is to immobilize the nucleic acid molecule, removing the impurities via washing and/or sedimentation techniques.

Optionally, therefore, the nucleic acid to be sequenced may be provided with a means for immobilization, which may be introduced during amplification, either through the nucleotide bases or the primer/s used to produce the amplified nucleic acid.

To facilitate immobilization, the amplification primers used according to the invention may carry a means for immobilization either directly or indirectly. Thus, for example the primers may carry sequences which are complementary to sequences which can be attached directly or indirectly to an immobilizing support or may carry a moiety suitable for direct or indirect attachment to an immobilizing support through a binding partner.

Numerous suitable supports for immobilization of DNA and methods of attaching nucleotides to them, are well known in the art and widely described in the literature. Thus for example, supports in the form of microtitre wells, tubes, dipsticks, particles, fibres or capillaries may be used, made for example of agarose, cellulose, alginate, teflon, latex or polystyrene. Advantageously, the support may comprise magnetic particles e.g. the superparamagnetic beads produced by Dynal Biotech ASA (Oslo, Norway) and sold under the trademark DYNABEADS. Chips may be used as solid supports to provide miniature experimental systems as described for example in Nilsson et al. (Anal. Biochem. (1995), 224:400-408).

The solid support may carry functional groups such as hydroxyl, carboxyl, aldehyde or amino groups for the attachment of the primer or capture oligonucleotide. These may in general be provided by treating the support to provide a surface coating of a polymer carrying one of such functional groups, e.g. polyurethane together with a polyglycol to provide hydroxyl groups, or a cellulose derivative to provide hydroxyl groups, a polymer or copolymer of acrylic acid or methacrylic acid to provide carboxyl groups or an amino alkylated polymer to provide amino groups. U.S. Pat. No. 4,654,267 describes the introduction of many such surface coatings.

Alternatively, the support may carry other moieties for attachment, such as avidin or streptavidin (binding to biotin on the nucleotide sequence), DNA binding proteins (e.g. the lac I repressor protein binding to a lac operator sequence which may be present in the primer or oligonucleotide), or antibodies or antibody fragments (binding to haptens e.g. digoxigenin on the nucleotide sequence). The streptavidin/biotin binding system is very commonly used in molecular biology, due to the relative ease with which biotin can be incorporated within nucleotide sequences, and indeed the commercial availability of biotin-labelled nucleotides. This represents one preferred method for immobilisation of target nucleic acid molecules according to the present invention. Streptavidin-coated DYNABEADS are commercially available from Dynal Biotech ASA.

As mentioned above, immobilization may conveniently take place after amplification. To facilitate post amplification immobilisation, one or both of the amplification primers are provided with means for immobilization. Such means may comprise as discussed above, one of a pair of binding partners, which binds to the corresponding binding partner carried on the support. Suitable means for immobilization thus include biotin, haptens, or DNA sequences (such as the lac operator) binding to DNA binding proteins.

When immobilization of the amplification products is not performed, the products of the amplification reaction may simply be separated by for example, taking them up in a formamide solution (denaturing solution) and separating the products, for example by electrophoresis or by analysis using chip technology. Immobilization provides a ready and simple way to generate a single-stranded template for the extension reaction. As an alternative to immobilization, other methods may be used, for example asymmetric PCR, exonuclease protocols or quick denaturation/annealing protocols on double stranded templates may be used to generate single stranded DNA. Such techniques are well known in the art.

The method of the present invention is particularly advantageous in the diagnosis of pathological conditions characterised by the presence of a particular or specific microorganism, particularly infectious diseases. The method can be used to characterise or type and quantify microbial (e.g. bacterial, protozoal and fungal) infections where samples of an infecting organism may be difficult to obtain or where an isolated organism is difficult to grow in vitro for subsequent characterisation (e.g. as in the case of P. falciparum or Chlamydia species). Due to the simplicity and speed of the method it may also be used to detect or identify a wide range of pathological agents which cause diseases such as of clinical importance. Even in cases where samples of the injecting organism may be easily obtained, the speed of this method compared with overnight incubation of a culture may make the method according to the invention preferable over conventional techniques.

The high capacity and convenience of the method also make it particularly suitable for screening large numbers of samples, or for screening for the presence of a large number of organisms. A large number of samples may be simultaneously analysed.

The invention also comprises kits for carrying out the method of the invention. These will normally include one or more of the following components:

optionally primer(s) for in vitro amplification; one or more primers for the primer extension reaction; nucleotides for amplification and/or for the primer extension reaction (as described above); a polymerase enzyme for the amplification and/or primer extension reaction; and means for detecting primer extension (e.g. means of detecting the release of pyrophosphate as outlined and defined above or means for detecting the incorporation of fluorescently labelled nucleotides).

In certain embodiments, the kit will also include instructions for the order of addition of the nucleotides.

The invention will now be described by way of non-limiting examples with reference to the drawings.

### EXAMPLE 1

### Materials and Methods

Bacterial Strains and DNA Extraction

The H. pylori reference collection of clinical isolates used in this example (HP-HJM 1-25) were obtained from routine clinical dyspeptic gastric biopsy specimens (mixed age and gender) at the University Hospital, Linköping. Reference strains H. pylori 26695 (CCUG 41936) and H. pylori J99 were obtained from the Culture Collection University of Gothenburg, Sweden, and Dr. L. Engstrand, SMI Stockholm, respectively. Bacteria were cultured as described elsewhere (Monstein, H. J., Kihlström, E. and Tiveljung, A. (1996) Detection and identification of bacteria using in-housing broad-range 16S rDNA PCR amplification and genus-specific hybridisation probes, located within variable regions of 16S rRNA genes. APMIS 104, 451-458). Genomic DNA from the H. pylori strains was prepared using a commercially available DNA extraction kit (QIAamp tissue kit, Qiagen, KEBO, Stockholm) as described (Monstein, H. J., Tiveljung, A. and Jonasson, J. (1998) Non-random fragmentation of ribosomal RNA in Helicobacter pylori during conversion to the cocoid form. FEMS Immunol. Med. Microbiol. 22, 217-224).

In Vitro Amplification of the 16S rRNA Gene

Primers used in this study (Table 1) were obtained from Amersham-Pharmacia Biotech Norden (Sollentuna, Sweden) or Scandinavian Gene Synthesis (Köping, Sweden). Sequential amplification of the 16S rRNA gene was performed using two sets of primer-pairs and Ready-To-Go® PCR Beads (Amersham-Pharmacia Biotech). The 16S rDNA variable V1 region was amplified using primers bio-pBR-5′/se (10 pmol) and pBR-V1/as (10 pmol), and the variable V3 region was amplified using primers bio-pJB-se and HP-V3T/as, respectively. PCR amplification was carried out in a thermal controller PTC-100™ (MJ Research Inc., SDS-Falkenberg) using 2 l of DNA extract and a final volume of 25 l as follows: denaturation step at 94° C. for 2 minutes (1 cycle); followed by denaturation at 94° C. for 40 seconds, annealing at 55° C. for 40 seconds, extension at 72° C. for 1 minutes (25 cycles) and a final extension step at 72° C. for 10 minutes. Subsequently, PCR amplified products (5 l) were analysed by agarose gel electrophoresis (Monstein H. J. et al., supra). The expected sizes for the V1 and V3 amplicons were approximately 110 bp and 85 bp, respectively.

TABLE 1
Primers used for PCR amplification and pyrosequencing.

| | | | |
|---|---|---|---|
| Primer name | Sequence (5′ to 3′ orientation | Position in | Tm (° C.)^{a} |

| | | | |
|---|---|---|---|
| bio-pBR-5′/se | biotin-GAAGAGTTTGATCATGGCTCAG | E. coli [12] | 48 |
| | | 6 | |
| pSR-V1/as | TTACTCACCCGTCCGCCACT | 120 | 51 |
| HP-V3T/as^{b} | AGCTCTGGCAAGCCAGACA | 1040 | 48 |
| bio-pJB-1/se | biotin-ATTCGATGCAACGCGAAGAACCTTACC | 960 | 55 |

^{a}The melting temperature was calculated according to the formula: Tm = 81.5 + 16.6 (log[K⁺]) + 0.41 (% GC) − (675/n), where [K⁺] = 0.050 M and n = chain length (13, 14)
^{b}H. pylori type strain CCUG 17874^{T} 16S rRNA variable V3 region (5)

Pyrosequencing™

Twenty μl of biotinylated V1 and V3 amplicons, respectively, were mixed with 25 l of 2× BW-buffer (10 mM Tris-HCl, 2 M NaCl, 1 mM EDTA and 0.1% Tween 20, pH 7.6) and 10 l Dynabeads (Dynabeads® M280-Streptavidin), and immobilised by incubation at 65° C. for 15 minutes (shaking). Single stranded DNA was obtained by incubation (1 minute) of the captured biotin-streptavidin complex (magnetic beads) in 50 l of 0.50 M NaOH (each well), using a PSQ 96 Sample Prep Tool (Pyrosequencing AB, Uppsala). Subsequently, each sample (well) was washed with 100 l 1X-annealing buffer (200 mM Tris-acetate and 50 mM Mg-acetate). pBR-V1/as (V1 region) and HP-V3T/as (V3 region of the type strain H. pylori CCUG 17874^{T}) , respectively, were also used as sequencing primers and hybridised to the single stranded PCR products. For that purpose, 1 l of sequencing primer (15 pmol) was incubated in 44 l of annealing buffer (each well) at 80° C. for 2 minutes, followed by cooling to room temperature. Pyrosequencing was performed using a SNP Reagent Kit (enzyme—and substrate mixture, dATP-S, dCTP, dGTP, and dTTP) as provided by the manufacturer (Pyrosequencing AB, Uppsala).

### Results

The present invention describes a new approach for rapid molecular identification and subtyping of H. pylori isolates by Pyrosequencing™ and signature matching of PCR-amplified variable regions within the 16S rDNA.

Partial sequences within the variable V1 and V3 regions were obtained from 25 strains of a H. pylori reference collection of clinical isolates and two reference strains (H. pylori 26695 and J99, respectively). One set of two primers was used for each locus (Table 1). Based on nucleotide sequences within the variable V1 region between positions 75 and 100, the 25 clinical isolates could be divided into six different lineages (FIG. 1). The corresponding Pyrograms™ are shown in FIG. 2. Lineage A comprising 11 isolates (HP-HJM 2,3,5,7,8,9,13,19,20,21,25) had a sequence that was identical with that of H. pylori 26695 (FIG. 1). Single or double nucleotide mutations were observed in lineages B (HP-HJM 1,4,14,18,22), C (HP-HJM 11,15,17,23) and D (HP-HJM 24) as compared with the H. pylori 26695 sequence (FIG. 1). A single nucleotide insertion was present in lineage E (HP-HJM 10). Lineage F (HP-HJM 6), which differed significantly in the V1 region from the other isolates, demonstrated DNA sequence identity with the corresponding region of reference strain H. pylori J99 (FIG. 1).

All isolates, except HP-HJM 10 and HP-HJM 21, revealed sequence identity in the V3-region (pyrograms not shown) with H. pylori CCUG 17874^{T}, H. pylori 26695, and H. pylori J99 (FIG. 1). HP-HJM 10 and HP-HJM 21 (lineages B and A, respectively, in the V1 region) demonstrated a single C to T transition (FIG. 1).

The short 25-30 nt DNA sequence obtained for each isolate and region was used as a “signature” of the 16S rDNA of the particular isolate, which thus gained multiple signature attributes. The uniqueness of each signature was investigated by matching it against a “signature template” consisting of all catalogued bacterial 16S rDNA sequences available at NCBI using the BLAST advanced option tools including taxonomy and lineage reports.

The primer HP-V3T/as used for sequencing between position 990 and 1020 of the V3 region was designed based on the H. pylori type strain CCUG 17874^{T} sequence (U01331). The Tax BLAST Lineage Report indicated specificity for Helicobacter group (epsilon subdivision of proteobacteria). Therefore, when HP-V3T/as is used as a primer in PCR, DNA from other microorganisms should in all likelihood not yield a PCR product under stringent conditions. Verification of the actual strain being a member of the species H. pylori was obtained for 23/25 isolates through the criteria of signature matching in the V3 region, disregarding the non-human Helicobacter nemestrinae (FIG. 1).

The primer pBR-V1/as used for sequencing between position 75 and 100 of the V1 region was designed as a broad-range primer based on conserved residues appearing in most clinically important eubacteria. The sequencing of the V1 segment was primarily aimed at allocating the actual strain to a certain lineage. However, despite the DNA sequence variation in this region, lineages A to E were tentatively identified as H. pylori also by signature matching of the V1 region allowing for one or two mismatches in those cases where the signature was unknown to the database. The H. pylori J99 and lineage F signatures of the V1 region matched with Helicobacter spp. such as H. bilis, H. hepaticus, H. canadensis, H. cinaedi, H. rappini, H. mustelae, and also with Campylobacter jejuni (FIG. 1).

In conclusion, the present findings show that subtle DNA sequence variation does occur in the 16S rDNA variable V1 and V3 regions of H. pylori, which provides a consistent system for subtyping. The PSQ96™ automated system allows for rapid (c. 30 min) determination of 20-30 nt of target sequences dispensed in 96-well microtiter plates. From the system output, information on nucleotide sequences could easily be extracted for automatic evaluation using a simple algorithm and a local 16S rDNA position based database.

### EXAMPLE 2

### Materials and Methods

Five hundred clinical isolates were collected from secretions, indwelling catheters and prosthetic devices, urine, blood and faecal specimens at the Laboratory Medicine Östergötland (LMÖ) microbiology unit, University Hospital, Linköping. Species with less than two isolates were excluded from the analysis. Based on calculations using VectorNTI (InforMix) and GenBank data, two sets of probes were selected with conserved motifs to be used as broad-range primers for PCR amplification of the V1 and V3 regions, respectively.

Clinical bacterial isolates were identified phenotypically using accredited standard methods at the LMÖ—Microbiology unit, University Hospital, Linköping. One colony (10 colonies if very small) of each isolate was suspended in a total of 100 μL Glycerol Broth (2.1% Nutrient broth No.2 (LabM) with 15% glycerol) and stored at −20 C.

Three primer sets (purchased from Scandinavian Gene Synthesis, Köping, Sweden) for broad-range PCR amplification and Pyrosequencing™ of 16S rDNA variable region V1 and V3 were used as follows:

To obtain V1 antisense Pyrosequencing™ product with start-position 100: 5′-biotinylated V1 sense primer bio-pBR5′.SE (position 6-27), 5′-GAAGAGTTTGATCATGGCTCAG-3′; V1 antisense primer pBR-V1.AS (position 120-101), 5′-TTACTCACCCGTCCGCCACT-3′; sequencing primer: pBR-V1.AS.

To obtain V3 antisense Pyrosequencing™ product with start-position 1027: 5′-biotinylated V3 sense primer bio-pJBS-V3.SE (position 947-967), 5′-GCAACGCGAAGAACCTTACC-3′; V3 antisense primer B-V3.AS (position 1047-1027), 5′-ACGACAGCCATGCAGCACCT-3′; sequencing primer: B-V3.AS.

To obtain V3 sense Pyrosequencing™ product with start-position 967: 5′-biotinylated V3 antisense primer bio-B-V3.AS (see above); V3 sense primer pJBS-V3.SE (see above); sequencing primer: pJBS-V3.SE.

PCR was carried out in 0.5 mL thin walled tubes with Ready-To-Go beads (Amersham-Pharmacia Biotech) and 5 pmol of each primer in 25 μL reaction volume. One μL of frozen bacterial suspension was added. A DNA thermal cycler PTC-100™ (M J Research Inc., SDS-Falkenberg) was used. After initial denaturation at 94 C for 10 min, 25 cycles of amplification were carried out starting at 94 C for 40 s, followed by 40 s at 55 C, and 60 s at 72 C. Final extension at 72 C for 10 min.

Pyrosequencing. Twenty μL of biotinylated PCR products were mixed with 10 μL Dynabeads M280-streptavidin solution (Dynal Biotech ASA, Norway) and 25 μL of 2×BW buffer pH 7.6 (10 mM Tris-HCl, 2M NaCl, 1 mM EDTA and 0.1% Tween 20) and incubated at 65 C for 15 min in a shaking mixer (1100 rpm). The immobilised biotinylated PCR products-streptavidin Dynabeads complex was captured using a PSQ96™ Sample Prep Tool. Strand separation of template DNA was obtained through incubation of the complex in 0.5M NaOH (50 μL per well) for 1 min followed by washing (by releasing and recapturing the beads) in 100 μL 1×Annealing buffer (200 mM Tris-acetate and 50 mM Mg-acetate). One μL of sequencing primer (15 pmol) was annealed to the immobilised template in 44 μL 1×Annealing buffer by heating at 80 C for 2 min followed by slow cooling to room temperature. For pyrosequencing a SNP Reagent Kit (dATP S, dCTP, dGTP, dTTP, enzyme and substrate mixtures) was used according to the instructions of the manufacturer (Pyrosequencing AB, Uppsala).

### Results and Discussion

In this example evidence is presented that the technique of the invention can be applied generally for provisional identification of clinically important bacteria.

The strategy to prove the feasibility of this approach was to perform verification analyses on a small number of each species of local routinely identified isolates of commonly encountered clinically important bacteria. The results indicate that the targeted motifs were sufficiently well conserved so that PCR amplicons representing V1 or V3 regions could be obtained as required for most relevant species. Pyrosequencing™ was performed from either end of the PCR products. The V1 antisense sequencing primer pBR-V1.AS targeted E. coli 16S rRNA position 120-101. The V3 region was sequenced in both directions. The V3 sense sequencing primer pJBS-V3.SE targeted E. coli 16S rRNA position 947-967, and the V3 antisense sequencing primer B-V3.AS targeted E. coli 16S rRNA position 1047-1027. FIG. 3 shows the pyrograms™ obtained. Automatic interpretation of the pyrograms™ was performed.

Using the pJBS-V3.SE sequencing primer all aerobic Gram-positive bacterial template sequences displayed A in the first position, whereas all those corresponding to aerobic Gram-negative bacteria had T in the first position (Table 2). Furthermore, using pBR-V1.AS sequencing primer and extending the analysis to three bases, all staphylococci had A, A, C in position 1, 2, and 3, respectively (Table 3). This triplet was sufficiently discriminative to designate a classification boundary of Staphylococcus against the other common isolates (Listeria monocytogenes excluded) (Table 3).

The sequence interpreted from the pyrograms™ was used as a signature of the 16S rDNA of the particular isolate and matched against the local database. The uniqueness of unknown signatures was investigated by matching them against all catalogued bacterial 16S rDNA sequences available at NCBI using the BLAST advanced option tools including taxonomy and lineage reports. As shown in Table 3, the first 10 bases following the pBR-V1.AS sequencing primer appears to be sufficient information to allow provisional species designation.

### Aerobic Gram-Positive Bacteria

Staphylococcus: Using the pBR-V1.AS sequencing primer all staphylococci could be provisionally identified by pyrosequencing ^{˜}10 nts (Table 3). This included all 28 routinely identified isolates of Staphylococcus aureus, the most virulent member of the genus frequently found in cutaneous and wound infections, septic arthritis, septicaemia etc, all 26 isolates of coagulase negative staphylococcus (CoNS), putative S. epidermidis found in prosthetic joint and catheter infections, and all 25 isolates of S. saprophyticus, a CoNS found in urinary tract infections.

Streptococcus: Using the pBR-V1.AS sequencing primer, all 26 isolates of Streptococcus pyogenes (group A), a common cause of pharyngitis and severe streptococcal toxic shock syndrome, all 25 isolates of Streptococcus agalactiae (group B), neonatal infections, and 30/30 isolates of Streptococcus pneumoniae, otitis media and pneumonia, were identified (Table 3). Equivalent results were obtained for the V3 region using the B-V3.AS sequencing primer.

Enterococcus: Using the pBR-V1.AS sequencing primer, all 25 isolates of E. faecalis, and all 16 isolates of E. faecium were identified (Table 3). Similarly, using the V3 sequencing primers, all 25 isolates of E. faecalis, and all 16 isolates of E. faecium were identified.

In conclusion, the aerobic Gram-positive bacteria investigated here all gave the expected PCR products without prior DNA extraction. The PCR amplicons could be used directly for Pyrosequencing™ and all isolates were accurately identified.

### Results for Aerobic Gram-Negative Bacteria

Enterobacteriaceae: Using the pBR-V1.AS sequencing primer, all 32 isolates of Escherichia coli, which is the species most commonly isolated, were provisionally identified on the first 10 bases. When using the pJBS-V3.SE sequencing primer, the E. coli isolates (starting with TGGT) could be readily separated from the E. cloacae isolates (starting with TACT) and also from the Salmonella isolates (Table 3).

Klebsiella, Enterobacter, Serratia, and Citrobacter are closely related genera. Using the pBR-V1.AS sequencing primer Klebsiella could be identified to genus but not differentiated into species. Using the pJBS-V3.SE sequencing primer, 30/43 Klebsiella isolates fitted the [KPY17668 (K. pneumoniae)] template starting with TGGT, whereas 13 isolates had a consensus sequence up to position 17 with [KOY17667 (K. oxytoca)] starting with TACT.

Proteus mirabilis is less closely related to E. coli. Using the pBR-V1.AS sequencing primer, notable homology up to position 28 was observed with Haemophilus influenzae. Better discrimination was achieved with the V3 sequencing primers (Table 3).

Haemophilus: Classification and provisional identification of 32 isolates of Haemophilus influenzae, which is a common cause of respiratory tract infections in children, was straightforward for all three primers (Table 3).

Pseudomonas: Using the pBR-V1.AS sequencing primer, all 30 isolates of P. aeruginosa causing skin infections and nosocomial infections (respiratory tract, wound infections, and septicaemia) had a sequence matching the AE004949 (P. aeruginosa PA01) template.

Thus, the aerobic Gram-negative bacteria investigated gave the expected PCR products and could be accurately identified although longer signatures and combining the results obtained for the V1 and V3 regions may be necessary for closely related species of enterobacteria.

TABLE 2

Signature templates using pJBS-V3.SE sequencing primer

| | | | |
|---|---|---|---|
| No.isol. | Phenotype | Sorted signature sequences <= 40 nts | Reference |

| | | | |
|---|---|---|---|
| 11 | Staphylococcus CoNS | AAATCTTGACATCCTCTGACCCCTCTAGAGATAGAGTTTT | ks74 (S. epid) |
| 14 | Staphylococcus CoNS | ----------------------TC----------------- | var2 |
| 26 | Staphylococcus saprophyticus | ---------------T---AAA--------------CC-- | L20250, NT75 |
| 29 | Staphylococcus aureus | ---------------T----AA--------CC-- | Y15856 |

*************** *** ************ **

| | | | |
|---|---|---|---|
| 2 | Fusobacterium spp | AGCGTTTGACATCCTACGAACGGAGCAGAGATGCGCCGGT | |
| 35 | Streptococcus pyogenes | AGGTCTTGACATCCGGATGCCCGCTCTAGAGATAGAGTTT | AF076028 |
| 31 | Streptococcus pneumoniae | ----------TC---A------------ | |
| 30 | Streptococcus agalactiae | ---------TTC--A---GC-------GC--- | JCM5671 |
| 2 | Enterococcus gallmnarum | ---------TT--A----A------- | |
| 16 | Enterococcus faecium | ---------TT--A--A----------C--\| | |
| 25 | Enterococcus faecalis | ---------TT--A---A----------C--\| | Y18293 |
| 2 | Listeria monocytogenes | ---------TT--A---A----G----C----C-- | |

************** ** ** ** ****

| | | | |
|---|---|---|---|
| 3 | Bacterioldes fragilis | CGGGCTTAAATTGCAGTGGAATGATGTGGAAACATGTCAG | |
| 2 | Clostridium perfringens | TACACTTGACATCCCTTGCATTACTCTTAATCGAGGAAAT | |
| 6 | Yersinia spp | TACTCTTGACATCCACGGAATTTAGCAGAGATGCTTTAGT | |
| 1 | Haemophilus parainfiuenzae | TACTCTTGACATCCAGAGAACATTCCAGAGATGGATTGG | |
| 10 | Enterobacter cloacae | -------------T-A-------T----T | ECY17665 |
| 8 | Klebsiella oxytoca | -------------T-AG------CT----T\| | KOY17667 |
| 5 | Klebsiella pneumoniae | -------------T-AG------CT----T\| | |
| 4 | Citrobacter freundil | -------------T-AG------CT----T\| | |
| 9 | Morganella morganii | -------------T-CAG--- | |
| 5 | Serratia spp | ---------------------T----------\| | |
| 7 | Enterobacter cloacae | -------------T------------T\| | AF157695 |
| 27 | Proteus mirabilis | --------------C---TCC-TT------A--GGA-T | AF008582 |
| 4 | Haemophilus parainfluenzae | -----------TG---TC--GT------ATGAGA-T | |
| 32 | Haemophilus influenzae | --------TA----G-GCT--------AGC-T-T | Rd rmA16S |

| | |
|---|---|
| ************* *** *** | U32755 |

| | | | |
|---|---|---|---|
| 4 | Clostridium spp | TAGACTTGACATCTCCTGOATTACTCTTAATCGAGGAAGT | |
| 7 | Acinetobacter spp | TGGCCTTGACATAGTAAGAACTTTCCAGAGATGGATTGGT | |
| 35 | Pseudomonas aeruginosa/spp | --------GC-G-------------- | PA01 |
| 2 | Stenotrophomonas maltophilia | --------GTCG-------------- | |

******** ********************

| | | | |
|---|---|---|---|
| 2 | Campylobacter jejuni | TGGGCTTGATATCCTAAGAACCTTATAGAGATATGAGGGT | AL139076 |
| 2 | Acinetobacter spp | TGGTCTTGACATAGTAAGAACTTTCCAGAGATGGATTGGT | |
| 3 | Moraxella catarrhalis | -------G--TC--G-----CGA | |

*************** **** ** ********

| | | | |
|---|---|---|---|
| 2 | Salmonella spp | TGGTCTTGACATCCACAGAACTTTCCAGAGATGGACTGGT | AF057362 |
| 4 | Escherichia coil | -----------------T----\| | O157:H7 rrsA |
| 5 | Klebsiella oxytoca | -------------T----\| | KPY17668 |
| 25 | Klebsiella pneumoniae | -------------T----\| | |
| 11 | Salmonella spp | --------------------GAA------------T-T-- | ST16SRD |
| 3 | Citrobacter spp | ------------------GA-GA---G-----------A | |
| 3 | Esoherichia coil | ------------------GA-GA--A--------ATGA | |
| 6 | Shigella spp | ------------------GA-GA----------- | |
| 2 | Shigella spp | ------------------G---G---T------AGAAT--\| | |
| 22 | Escherichia coil | ------------------G---G---T------AGAAT--\| | O157:H7 rrsG |

**************** * * ******

| | | |
|---|---|---|
| 8 | Neisseria gonorrhoeae | TGGTTTTGACATGTGCGGAATCCTCCGGAGACGGAGGAGT |
| 481 | | |

Footnotes:
1) Asterix marks alignment within a group
2) Vertical double lines delineate sequences in consensus

TABLE 3
Signatures vs. phenotype using sequencing primer pBR-V1.AS

| V1.as | Phenotype | | | | | | | | | | | |
|---|---|---|---|---|---|---|---|---|---|---|---|---|
| First 10 bases | Staphylococcus | Staphylococcus | Staphylococcus | Listeria | Acinetobacter | Moraxella | Clostridium | Clostridium | Fusobacterium | Streptococcus | Streptococcus | Enterococcus |
| using pBR-V1.AS | aureus | saprophyticus | coNS | monocytogenes | spp. | catarrhalis | perfringens | spp. | spp. | agalactiae | pneumoniae | faecalis |
| AACATCAGAG | 28 | | | | | | | | | | | |
| AACGTCAAAG | | 26 | | | | | | | | | | |
| AACGTCAGAG | | | 25 | | | | | | | | | |
| AACTTTGGAA | | | | 2 | | | | | | | | |
| AAGATCAGTA | | | | | 4 | | | | | | | |
| AAGTATCAGA | | | | | | 4 | | | | | | |
| AATCCTTCCG | | | | | | | 2 | | | | | |
| AGATTTGTTC | | | | | | | | 4 | | | | |
| CAAGTCCGAA | | | | | | | | | 2 | | | |
| CATCAGTCTA | | | | | | | | | | 25 | | |
| CATCCAGAGA | | | | | | | | | | | 30 | |
| CCTCTTTCCA | | | | | | | | | | | | 25 |
| CCTCTTTTTC | | | | | | | | | | | | |
| CCTTGAACCG | | | | | | | | | | | | |
| CGCCACCCAA | | | | | | | | | | | | |
| CGCCACCCGA | | | | | | | | | | | | |
| CGCCGGCAAA | | | | | | | | | | | | |
| CGTCACCCAA | | | | | | | | | | | | |
| CGTCACCCAG | | | | | | | | | | | | |
| CGTCACCCGA | | | | | | | | | | | | |
| CGTCAGCAAA | | | | | | | | | | | | |
| CGTCAGCAAG | | | | | | | | | | | | |
| CGTCAGCAGA | | | | | | | | | | | | |
| CGTCAGCGAA | | | | | | | | | | | | |
| CGTCATCAAA | | | | | | | | | | | | |
| CTCAAGAGAA | | | | | | | | | | | | |
| CTTTCTTCGG | | | | | | | | | | | | |
| GAATCCAGGA | | | | | | | | | | | | |
| | 28 | 26 | 25 | 2 | 4 | 4 | 2 | 4 | 2 | 25 | 30 | 25 |

| V1.as | Phenotype | | | | | | | | | | | | |
|---|---|---|---|---|---|---|---|---|---|---|---|---|---|
| First 10bases | Enterococcus | Streptococcus | Stenotrophomonas | Neisseria | Yersinia | Citrobacter | Serratia | Klebsiella | Klebsiella | Enterobacter | Pantoea | Enterobacter | Salmonella |
| using pBR-V1.AS | faecium | pyogenes | maltophilia | gonorrhoeae | spp. | freundii | spp. | oxytoca | pneumoniae | cloacae | agglomerans | aeroganes | spp. |
| AACATCAGAG | | | | | | | | | | | | | |
| AACGTCAAAG | | | | | | | | | | | | | |
| AACGTCAGAG | | | | | | | | | | | | | |
| AACTTTGGAA | | | | | | | | | | | | | |
| AAGATCAGTA | | | | | | | | | | | | | |
| AAGTATCAGA | | | | | | | | | | | | | |
| AATCCTTCCG | | | | | | | | | | | | | |
| AGATTTGTTC | | | | | | | | | | | | | |
| CAAGTCCGAA | | | | | | | | | | | | | |
| CATCAGTCTA | | | | | | | | | | | | | |
| CATCCAGAGA | | | | | | | | | | | | | |
| CCTCTTTCCA | | | | | | | | | | | | | |
| CCTCTTTTTC | 16 | | | | | | | | | | | | |
| CCTTGAACCG | | 26 | | | | | | | | | | | |
| CGCCACCCAA | | | 4 | | | | | | | | | | |
| CGCCACCCGA | | | | 8 | | | | | | | | | |
| CGCCGGCAAA | | | | | 6 | | | | | | | | |
| CGTCACCCAA | | | | | | 5 | | | | | | | |
| CGTCACCCAG | | | | | | | 3 | | | | | | |
| CGTCACCCGA | | | | | | | | 13 | 30 | 2 | 3 | 4 | |
| CGTCAGCAAA | | | | | | | | | | 5 | | | 14 |
| CGTCAGCAAG | | | | | | | | | | | | | |
| CGTCAGCAGA | | | | | | | | | | | | | |
| CGTCAGCGAA | | | | | | | | | | 8 | 2 | | |
| CGTCATCAAA | | | | | | | | | | | | | |
| CTCAAGAGAA | | | | | | | | | | | | | |
| CTTTCTTCGG | | | | | | | | | | | | | |
| GAATCCAGGA | | | | | | | | | | | | | |
| | 16 | 26 | 4 | 8 | 6 | 5 | 3 | 13 | 30 | 15 | 5 | 4 | 14 |

| V1.as | Phenotype | | | | | | | | | | |
|---|---|---|---|---|---|---|---|---|---|---|---|
| First 10 bases | Escherichia | Haemophilus | Proteus | Morganella | Haemophilus | Shigella | Citrobacter | Enterococcus | Pseudomonas | Pseudomonas | |
| using pBR-V1.AS | coli | influenzae | mirabilis | morganii | parainfluenza | spp. | diversus | gallinarum | aeruginosa | spp. | |
| AACATCAGAG | | | | | | | | | | | 28 |
| AACGTCAAAG | | | | | | | | | | | 26 |
| AACGTCAGAG | | | | | | | | | | | 25 |
| AACTTTGGAA | | | | | | | | | | | 2 |
| AAGATCAGTA | | | | | | | | | | | 4 |
| AAGTATCAGA | | | | | | | | | | | 4 |
| AATCCTTCCG | | | | | | | | | | | 2 |
| AGATTTGTTC | | | | | | | | | | | 4 |
| CAAGTCCGAA | | | | | | | | | | | 2 |
| CATCAGTCTA | | | | | | | | | | | 25 |
| CATCCAGAGA | | | | | | | | | | | 30 |
| CCTCTTTCCA | | | | | | | | | | | 25 |
| CCTCTTTTTC | | | | | | | | | | | 16 |
| CCTTGAACCG | | | | | | | | | | | 26 |
| CGCCACCCAA | | | | | | | | | | | 4 |
| CGCCACCCGA | | | | | | | | | | | 8 |
| CGCCGGCAAA | | | | | | | | | | | 6 |
| CGTCACCCAA | | | | | | | | | | | 5 |
| CGTCACCCAG | | | | | | | | | | | 3 |
| CGTCACCCGA | | | | | | | | | | | 52 |
| CGTCAGCAAA | 32 | | | | | | | | | | 51 |
| CGTCAGCAAG | | 34 | 24 | | | | | | | | 58 |
| CGTCAGCAGA | | | | 9 | 3 | | | | | | 12 |
| CGTCAGCGAA | | | | | | 8 | 3 | | | | 21 |
| CGTCATCAAA | | | | | 1 | | | | | | 1 |
| CTCAAGAGAA | | | | | | | | | | 1 | 1 |
| CTTTCTTCGG | | | | | | | | 2 | | | 2 |
| GAATCCAGGA | | | | | | | | | 30 | 4 | 34 |
| | 32 | 34 | 24 | 9 | 3 | 8 | 3 | 2 | 30 | 5 | 477 |

### EXAMPLE 3

### Real-Time Sequencing of Regions Within the RNase P Gene for Typing Purpose

Background

The RNase P gene rnpB found in all bacteria can be used for typing purposes. The approximately 400 bp gene is present in only one copy in the genome and is transcribed to a catalytic RNA involved in tRNA processing. The RNase P gene contains both highly conserved regions and highly variable regions. The mixture of conserved and variable sequences and the small size of this gene make this gene especially well suited for sequence analysis and typing purposes. Specific oligonucleotides are hybridised to the conserved regions within the gene and the real-time sequencing reaction is directed into the variable sequences.

Some regions of the rnpB gene are especially attractive for typing purposes, e.g. regions P3 and P19. In bacteria, the region analyzed could vary depending on which bacterial species is targeted. The DNA from the bacteria is released by standard proteinase K treatment and used in an initial PCR amplification step where the rnpB gene is amplified.

Materials and Methods

In the case of Chlamydiaceae e.g. JB1 5′-CGA ACT AAT CGG AAG AGT AAG GC-3′ and JB2 5′-GAG CGA GTA AGC CGG (A/G) TTC TGT-3′ were used to generate an approximately 400 bp long DNA fragment using standard PCR conditions and reagents. Either of the two primers can be biotinylated for convenient sample preparations of the single-stranded DNA template used in the real-time reaction. The sequencing primers used in the Pyrosequencing™ reaction targeted the P3 region of Chladydiaceae RNase P RNA gene.

(oligonucleotide MK Forward: 5′- AAG AGT AAG GCA (A/G)CC GC-3′ and MK2 Reverse: 5′- AGT CC(G/T) GAC TTT CCT CT-3′). The variable region P19 is targeted with primers MK3 Forward: TAG A(T/G)G AAT G(G/A) (T/C)TGC and MK4 Reverse: TAA GCC GGU TTC TGT C-3′. The sequence obtained by the real-time reaction, reagents and instruments commercial available by Pyrosequencing AB, Sweden following protocol by the company, was then compared with the available RNase P RNA gene database generated on the compiled DNA sequences of the RNase P gene.

An algorithm can be applied to the sequence result to determine the discriminatory power of the reaction. Additional regions of the RNase P gene (e.g. P12 and P17) can be analysed in the real-time sequencing reaction to increase the discriminatory power of the assay.

I claim:

1. A method of identifying a microorganism in a sample, said method comprising:
determining the sequence of a region of up to 50 nucleotides in a predetermined site in a gene of said microorganism, thereby to obtain a signature sequence; and
analysing sequencing information in said signature sequence to identify said microorganism,
wherein said sequence is determined by detecting the nucleotides incorporated in a primer extension reaction performed using a primer binding at a predetermined site in said gene.

2. The method of claim 1 wherein said gene is an RNA gene.

3. The method of claim 1 wherein said gene encodes the RNA components of telomerases, splicesomes and/or other RNA-protein complexes.

4. The method of claim 2 wherein said RNA gene is a ribosomal RNA (rRNA gene).

5. The method of claim 4 wherein the rRNA gene is 5S rRNA, 16S rRNA, 18S rRNA, 23S rRNA and/or 26S rRNA.

6. The method of claim 5 wherein the rRNA gene is the 16S rRNA gene.

7. The method of claim 6 wherein said predetermined site in the 16S rRNA gene is selected from one or more of the nine variable regions, V1 to V9.

8. The method of claim 2 wherein said gene is a ribozymal RNA gene.

9. The method of claim 8 wherein the ribozymal RNA gene is the RNA component of RNase P.

10. The method of claim 9 wherein said predetermined site is selected from one or more of the variable regions P3, P12, P17 and P19 loops.

11. The method of claim 1 wherein the region sequenced is 10 to 40 nucleotides long.

12. The method of claim 1 wherein the region sequenced is 10 to 15 nucleotides long.

13. The method of claim 1 wherein the pre-determined primer binding site lies in a conserved or semi-conserved region.

14. The method of claim 1 wherein one or more further regions of up to 50 nucleotides of a gene are sequenced.

15. The method of claim 1 wherein the primer extension reaction is performed by sequentially adding nucleotides in a predetermined order of addition in the presence of a polymerase.

16. The method of claim 1 wherein as each nucleotide is added, it is determined whether or not the nucleotide is incorporated into the extended primer by the polymerase.

17. The method of claim 3 wherein as each nucleotide is added, it is determined whether or not the nucleotide is incorporated into the extended primer by the polymerase.

18. The method of claim 9 wherein the nucleotide incorporation is detected by detecting PPi release.

19. The method of claim 1 wherein the strain of said microorganism is identified.
